(19) United States
(12) Patent Application Publication
(10) Pub. No.: US 2007/0122831 A1
(43) Pub. Date: May 31, 2007

(54) Detection and source identification of microbial contaminants in water samples

(76) Inventor: Dave Bachoon, Milledgeville, GA (US)

Correspondence Address: KING & SPALDING LLP, 1180 PEACHTREE STREET, ATLANTA, GA, 30309-3521, US

(21) Appl. No.: 11/599,163

(22) Filed: Nov. 13, 2006

Related U.S. Application Data
(60) Provisional application No. 60/735,282, filed on Nov. 12, 2005.

(51) Int. Cl. C12Q 1/68

(52) U.S. Cl. 435/6

(57) ABSTRACT

The present invention relates generally to methods of microbial source tracking in environmental water samples. More specifically the present invention relates to methods of microbial source tracking using detection of Bifidobacterium species as markers of source contamination in environmental water samples. The present invention utilizes differences in DNA sequence between genes common to all Bifidobacterium as a means for detecting which species are present in an environmental water sample. This species specific information can then be used to determine the source of fecal contamination.

### CROSS-REFERENCE TO RELATED APPLICATIONS

The present application claims the benefit of U.S. Provisional Patent Application Ser. No. 60/735,282, filed Nov. 12, 2005, and is incorporated herein by reference in its entirety.

### FIELD OF INVENTION

This invention relates generally to methods of microbial source tracking. More specifically, the methods of this invention relate to determining the source of fecal contamination using molecular biology based techniques for the detection of Bifidobacterium species in environmental water samples.

### BACKGROUND OF THE INVENTION

Coastal environments are constantly threatened by pollution. Estuaries, which are coastal bodies of water that have access to both salt water from an ocean and fresh water from a river, are one of these threatened environments. Estuaries are fragile ecosystems that provide habitat and nursery for many important commercial and non-commercial species of marine life. These habitats, however, are threatened by waters polluted with the microbial contaminants found in the feces of both humans and animals (Bernhard et al. Appl. Environ. Microbiol. (2000) 66:1587-1594). Shellfish aquaculture and the harvest of wild clams and oysters are threatened by fecal contamination (Crane et al. Environ. Manag. (2003) 1(4):141-151, Nebra et al. Appl. Environ. Microbiol. (2003) 69(5):2651-2656). This causes a severe and widespread negative economical impact on local and state revenues as well as on human health. The pollution of coastal waters can impact human safety by creating unsafe drinking water, by initiating beach health advisories, and by precipitating the closing of beaches to the public (Scott et al. Appl. Environ. Microbiol. (2002) 68:5796-5803). In addition, fecal bacteria in water from humans and animals transported by coastal waters can transmit various diseases, such as typhoid fever and hepatitis (Cabelli. Water Sci. Technol. (1983) 15:1-15). Fecal contamination can originate from a variety of sources including faulty septic tanks, wildlife, and agricultural runoff (Cabelli (1983), Griffith et al. J. Wat. Health (2003) 1(4):141-151, Nebra (2003), Wheeler et al. J. Environ. Qual. (2002) 31:1286-1293). Therefore, determining the exact origin of the fecal pollution is important for preventing and managing the problem of microbial contamination.

Many different approaches have been used to determine the sources of water pollution. All of these methods, however, have specific drawbacks. They either are inconsistent, take too much time to perform, or do not give specific results as to the actual source of the pollution. For example, REP-PCR is the use of PCR to obtain DNA fingerprints from bacteria to determine the origin of fecal pollution (Carson et al. Appl. Environ. Microbiol. (2003); 69:1836-1839). Large numbers of fingerprints, however, from different bacteria are needed to build a library to compare to the fingerprints found in water samples. Also, the same bacteria from one region of the country may not produce the same fingerprints as bacteria found in another part of the country. A fingerprint library from one area would therefore not be usable in another, making this method time consuming, expensive and often inaccurate.

Other methods are unable to distinguish the sources of the pollution. Recently, the EPA has adopted Enterococcus as an indicator of fecal coliforms found in the water. This method has proven to be reliable and there is a strong correlation between enterococci numbers in the water and swimmer associated gastrointestinal illness (U.S. Environmental Protection Agency (1997) Method 1600, EPA 821/R-97/004. Office of Water, Washington D.C.). This method, however also has flaws. It is only able to determine whether a river is polluted; it is unable to distinguish between the sources of the pollution and provides no help in identifying the origin of the pollution. For these and other reasons, a novel bacterium has been sought which would be able to adequately distinguish the feces of different animals in water (Bonjoch et al. Appl. Environ. Microbiol. (2004) 70:3171-3175).

### BRIEF DESCRIPTION OF THE DRAWINGS

FIG. 1 shows the detection of B. adolescentis in a serial dilution of sewage samples. Lane 1=2 kb Ladder, Lane 2=Negative Control, Lane 3=B. adolescentis genomic DNA, Lane 4=Sewage DNA, Lane 5- 1/10 Sewage Dilution, Lane 6= 1/100 Sewage Dilution, Lane 7= 1/1000 Sewage Dilution, Lane 8=2 kb Ladder.

FIG. 2 shows detection of PCR amplification product using 785R (SEQ ID NO:1) and IM26F primers (SEQ ID NO:2). Lane 1=2 kb Ladder, Lane 2=Cow Feces, Lane 3=B. adolescentis genomic DNA, Lane 4=DNA extracted from 1/1000 sewage dilution, Lane 5=Altamaha River Sound, Lane 6=West Point—Fedrica River, Lane 7=Dunbar Creek, Lane 8=2 kb Ladder.

FIG. 3 shows detection of B. adolescentis in environmental water samples. Lane 1=2 kb Ladder, Lane 2=Cow Feces, Lane 3=B. adolescentis genomic DNA, Lane 4=DNA extracted from 1/1000 sewage dilution, Lane 5=Altamaha River Sound, Lane 6=West Point—Fedrica River, Lane 7=Dunbar Creek, Lane 8=2 kb Ladder

FIG. 4 shows DNA-DNA hybridization results when using a dog bacterial 16S rDNA Probe. Lane 1=Cow Fecal DNA, Lane 2=Dog Fecal DNA, Lane 3=Horse Fecal DNA, Lane 4=Goose Fecal DNA.

FIG. 5 shows DNA-DNA hybridization results when using a dog bifidobacteria 16S rDNA Probe. Lane 1=Dog Fecal DNA, Lane 2=Cow Fecal DNA, Lane 3=Horse Fecal DNA, Lane 4=Goose Fecal DNA, Lane 5=Chicken Fecal DNA, Lane 6=Pig Fecal DNA.

FIG. 6 shows DNA-DNA hybridization results when using a pig bifidobacteria 16S rDNA Probe—Lane 1=Dog Fecal DNA, Lane 2=Cow Fecal DNA, Lane 3=Horse Fecal DNA, Lane 4=Goose Fecal DNA, Lane 5=Chicken Fecal DNA, Lane 6=Pig (1) Fecal DNA, Lane 7=Pig (2) Fecal DNA.

### SUMMARY OF THE INVENTION

Bifidobacteria species can be used as indicators for the source of fecal contamination in water samples (Bonjoch (2004), Gueimonde et al. Appl. Environ. Microbiol. (2004) 70:4165-4169, Long et al. Can. J. Microbiol. (2005) 51(5):413-422, Mullie et al. FEMS Microbiol Lett. (2003) 222(1):129-136). Bifidobacteria are gram-positive rods and strict anaerobes. These bacteria are difficult to culture have strict growing conditions. They are, however excellent candidates for the discrimination of fecal contamination because they make up a significant portion of the microflora present in the intestines of humans and animals. They are also regularly shed in the feces.

When animal waste is introduced into water environments, the DNA from Bifidobacterium species can be detected. Different species of Bifidobacterium found in the feces of certain animals can be used to identify the source of contamination. For example, B. adolescentis and B. dentium are found only in the intestines of humans. Differences in the DNA sequence of the 16S rRNA gene, tansaldolase gene and GRE gene, among other common genes shared by all Bifidobacterium species, can be use to distinguish one species from the other. Likewise, detection of a Bifidobacterium species known to be specific to one animal can then be used to determine the source of fecal contamination. In one embodiment, the present invention may be used to detect the presence of human fecal contamination in environmental water samples. For example, bacteria in the water sample may be extracted by capture on a solid adsorbent and then lysed directly on the solid adsorbent followed by isolation of the bacterial DNA. The isolated DNA in the previous step may then be used as a template for the amplification of a portion of the 16S rRNA gene specific to all Bifidobacterium species. The amplified DNA from this step may then be used as the template for the amplification of a portion of the 16S rRNA gene specific, for example, to B. adolescentis. Detection of this B. adolescentis specific portion of the 16S rRNA gene then indicates the presence of human fecal contamination in the water sample.

In another embodiment, the present invention may be used to determine the source of fecal contamination in an environmental water sample. For example, bacteria in the water sample may be extracted by capture on a solid adsorbent and then lysed directly on the solid adsorbent followed by isolation of the bacterial DNA. The isolated DNA in the previous step may then be amplified and labeled with a detectable marker. This labeled and amplified probe DNA may then be hybridized to a membrane containing an array of DNA isolated from the feces of various animal species to be tested. Hybridization of the labeled probe DNA to the fecal DNA of a given species indicates the source or sources of fecal contamination in the water sample.

### DETAILED DESCRIPTION OF THE INVENTION

One advantage of the present invention is that it uses a culture independent approach for the rapid detection of Bifidobacterium species DNA in order to determine the source of fecal pollution in environmental water samples. Methods for detecting Bifidobacterium spp. in municipal sewage and animal waste water have been developed, however, a method for detecting Bifidobacterium spp. as markers of fecal contamination in environmental water sources has not previously been shown to be effective. In certain embodiments, for example, the present method can detect and determine the source of Bifidobacterium spp. in water samples having an enterococcal count of approximately 22 CFU/100 ml within 24 hours.

Isolation of Bacteria

Although several methods of isolating bacteria may be compatible with the present invention, the isolation of bacteria from the environmental water sample is preferably performed by capturing the bacteria on a solid adsorbent. Isolation of bacteria according to certain embodiments of this invention may be obtained by filtration of the water sample through a filter membrane with a pore size of 0.20-0.44 μm. More preferably, a filter membrane with a pore size of 0.22 μm can be used. In such preferred embodiments, the pore size should be sufficiently small so that bacteria are prevented from flowing through the filter and instead become trapped on the filter. The filter material may be, but is not limited to, nitrocellulose, cellulose, polycarbonate, Teflon, nylon, polycarbonate, polyester, polyethersulfone, or polypropylene. Appropriate housing for the membrane filter will generally be determined by the sample volume to be processed; shape of the filter and size or diameter of the filter and can be determined by one of ordinary skill in the art. Filtration can be conducted under appropriate atmospheric pressure or under vacuum. In order to ensure sufficient capturing of the bacteria on the filter, a flow rate in the range of 20-80 cm/Hg, should be considered. More preferably, a flow rate of 40 cm/Hg can be used. Sample volumes in the range of 0.5 ml to more than 1 L are compatible with the current method, but smaller or larger volumes may be suitable. In one embodiment, a sample volume of 100 mls is used. In other embodiments of the present invention, multiple filter membranes may also be employed in the filtration step. For example, a larger pore size membrane may be stacked on top of a smaller pore size membrane in order to trap larger particulate matter and other elements that may interfere with the downstream extraction and isolation of bacterial DNA. The membrane filters may comprise the same or different materials.

Isolation of Bacterial DNA

Isolation of bacterial DNA can be accomplished using any number of DNA extraction methods known in the art. For example, isolation of bacteria DNA cell lysis may be effected by brief exposure to extremes of pH, organic solvents, chaotropic agents like urea and guanidine HCl, detergents like sodium dodecyl sulfate (SDS) and Triton X-100, osmotic shock, lysozyme digestion, protease digestion, or the use of shear forces. The DNA can be separated from lysed cellular debris and other potential interfering substances through such means as organic solvent extraction, acid precipitation, ultrafiltration, solid-phase extraction, HPLC, LiCl precipitation, protease digestion, RNase digestion, or polyethylene glycol precipitation and the like. In one embodiment, the DNA may be extracted using a modification of the “Alternative Protocol” of the UltraClean™ Soil DNA kit (MO BIO Laboratories, Carlsbad, Calif., Product # 12800). For example, the filter containing the trapped bacteria can be placed in a Petri dish. The bead solution may be initially separated from the beads and placed in the Petri dish containing the filter. Solutions S1 and IRS may then be added directly to the Petri dish. In such embodiments, the amounts of solutions S1 (MO BIO's lysis solution) and IRS (MO BIO's inhibitor removal solution) are preferably added in the proportions indicated by the kit's protocol and sufficient to completely cover the filter membrane. The dish is then vortexed vigorously for 5-30 minutes. The solution can then be removed and returned to the bead tubes. The reminder of the isolation step follows the manufacture's protocol, or protocols known to those of ordinary skill in the art.

Nested PCR for Detection of B. adolescentis

A nested PCR protocol should be used to detect B. adolescentis in environmental water samples. In such a protocol, the first step should comprise amplification of the isolated bacterial DNA using a universal eubacteria 16S rRNA primer in combination with a Bifidobacterium genus specific primer. Suitable primer pairs can be determined by one of skill in the art using such tools as BLAST and any number of readily available primer design programs such as Primer3 (Steve Rosen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386). An example of a preferred universal eubacteria primer is 785R (SEQ ID NO: 1) and an example of a preferred Bifidobacterium genus specific primer is IM26F (SEQ ID NO: 2).

The PCR reaction can be performed using any number of commercially available PCR kits and protocols available and know in the art. In one preferred embodiment, the PCR reaction is performed in 50 μl volume with 0.3 mM dNTP, 4 mMgCl₂, 1.5 U Taq DNA polymerase, 1×PCR reaction buffer, and approximately 30 ng of template DNA. The concentrations of all of the above components can be varied to further optimize the PCR reactions, if needed, and can be determined by one of ordinary skill in the art. In addition, PCR enhancing agents such as DMSO, betaine, formamide, glycerol, nonionic detergents, bovine serum albumin, polyethylene glycol, tetramethylammonium chloride and the like can be added to further increase yield, specificity, and consistency as needed. The PCR reaction can be run on any suitable PCR thermocycler. The choice of denaturation, annealing and extension temperatures, the length of time for each step in a thermal cycle, and the total number of cycles can be determined by one of ordinary skill in the art and will be specific to the primers used and the target sequence to be amplified. In a PCR reaction utilizing the 785R and IM26F primer pair, the following conditions can be used: initial denaturing at 94° C. for 5 minutes; 30 cycles of 94° C. for 30 seconds, 48° C. for 30 seconds, and 72° C. for 30 seconds; and final elongation at 72° C. for 5 minutes. The amplified product from this PCR reaction is then used as the template for a second PCR mixture For the second PCR reaction, the template from the first reaction is amplified using B. adolescentis species specific primers. The choice and design of the primers can be determined by one of ordinary skill in the art using the methods referred to above. An example of a preferred B. adolescentis specific primer pair is ADO1 (SEQ ID NO: 3) and ADO2 (SEQ ID NO: 4) (6). A volume of 1 to 5 μl of the first PCR reaction can be used. The PCR reaction can be performed using any number commercially available kits and protocols. In one embodiment, 1 μl of PCR product from the first reaction was added to added to a 50 μl reaction mixture containing the same concentrations of MgCl₂, reaction buffer, dNTP, and Taq polymerase as used in the first reaction. PCR enhancing agents like those listed above may also be added to the reaction as needed. The choice of denaturation, annealing and extension temperatures, the length of time for each step in a thermal cycle and the total number of cycles can be determined by one of ordinary skill in the art and will be specific to the primers used and the target sequence to be amplified. In a PCR reaction utilizing the ADO1 and ADO2 primer pair, the following conditions can be used: initial denaturing at 94° C. for 5 minutes; 45 cycles of 94° C. for 30 seconds, 48° C. for 30 seconds, and 72° C. for 30 seconds; and final elongation at 72° C. for 5 minutes.

The products of both PCR reactions can be analyzed by gel electrophoresis on polyacrylamide or agarose gels or other suitable medium and visualized by staining using appropriate staining agents such as ethidium bromide. In certain embodiment of the present invention, the PCR product amplified using primers 785R and IM26F result is 777 bp and the product amplified by the ADO1 and ADO2 primer pair is 279 bp. The above method is also compatible with real time PCR and can be modified for such by one of ordinary skill in the art using any number of commercially available kits and real time PCR thermocylers. The use of real time PCR has the advantage of not only detecting B. adolescentis, but also providing a rough approximation of the amount of B. adolescentis in the sample (M.W. Pfaffl Nucleic Acids Res. 2001, 29(9):e45). The detection of B. adolescentis indicates 15 the presence of human fecal contamination in the water sample. The above PCR reactions can be combined into a single reaction, however, previous experiments have shown improved sensitivity and consistency when the nested PCR is conducted as two separate reactions.

DNA-DNA Hybridization for Source Determination of Fecal Contamination

Probe DNA may be extracted from bacteria isolated from water samples as described above under “Isolation of Bacteria” and “Isolation of Bacterial DNA.” Target DNA is obtained from the feces of potential animal sources of fecal contamination. Typical sources of fecal contamination to be examined with this method include, but are not limited to, human, cow, horse, goat, pig, goose, and chicken. The DNA can be obtained from any animal fecal matter representative of the potential source of contamination. The DNA does not have to be extracted from the feces of the animals directly believed to be the source of fecal contamination. Likewise, the DNA can be extracted in bulk quantities and aliquoted and properly stored for latter use. Extraction of fecal DNA can be performed using methods known in the art or using commercially available kits such as the UltraClean™ Fecal DNA Kit (MO BIO Laboratories, Carlsbad, Calif. Product # 12811).

Probe DNA and target DNA may be amplified in separate reactions using a universal eubacteria-specific primer and a Bifidobacterium genus specific primer. Suitable primer pairs can be determined by one of skill in the art using such tools as BLAST and any number of readily available primer design programs such as Primer3 (Steve Rosen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386). An example of a preferred universal eubacteria primer is 785R (SEQ ID NO: 1) and an example of a preferred Bifidobacterium genus specific primer is IM26F (SEQ ID NO: 2). Another preferred primer pair for target and probe preparation is GRE-UNI (SEQ ID NO:7) and GRE-REV (SEQ ID NO:8) The PCR reaction can be performed using any number of commercially available PCR kits and protocols.

In a preferred embodiment, the PCR reaction is performed in 50 μl volume with 0.3 mM dNTP, 4 mMgCl₂, 1.5 U Taq DNA polymerase, 1×PCR reaction buffer, and approximately 30 ng of template DNA. The concentrations of all of the above components can be varied to further optimize the PCR reactions if needed and can be determined by one of ordinary skill in the art. In addition, PCR enhancing agents such as DMSO, betaine, formamide, glycerol, nonionic detergents, bovine serum albumin, polyethylene glycol, tetramethylammonium chloride and the like can be added to further increase yield, specificity and consistency as needed. The PCR reaction can be run on any suitable PCR thermocycler. The choice of denaturation, annealing and extension temperatures, the length of time for each step in a thermal cycle, and the total number of cycles can be determined by one of ordinary skill in the art and is specific to the primers used and the target sequence to be amplified. In a PCR reaction utilizing the 785R and NM26F primer pair, the following conditions are preferably used: initial denaturing at 94° C. for 5 minutes; 30 cycles of 94° C. for 30 seconds, 48° C. for 30 seconds, and 72° C. for 30 seconds; and final elongation at 72° C. for 5 minutes.

Probe DNA may be labeled with a detectable marker using methods known in the art. The probe can be labeled with, but not limited to, colorimetric dyes, fluorescent dyes, and radioactive isotopes. In a preferred embodiment, the probe is labeled with digoxigenin by adding 0.15 μl of 25 nmol digoxigenin-11-UTP to the PCR reaction mixture.

Target DNA may be fixed to an acceptable hybridization membrane using methods known in the art. Suitable membrane substrates include, but are not limited to, nylon and nitrocellulose. The amount of target DNA fixed to the membrane can be from 1 ng-1 μg. In a preferred embodiment, samples of target DNA from each animal source to be screened are boiled for 5-10 minutes and then immediately placed in an ice bath. Then long of target DNA from each animal source to be screened are spotted on a separate and distinct spot on the membrane. The membrane can then be cross-linked in a UV crosslinker at 1.2×10⁵ J/cm². The present invention is also compatible with microarray formats. Appropriate amounts and methods of spotting fecal DNA on microchips can be done using methods known in the art. Solid microchip substrates that can be used with the present invention, include but are not limited to, glass; coated class, including by not limited to, glass coated with, epoxy silane, aminosilane, aldehyde silane, poly-L-lysine; metallized coatings; nitrocellulose and nylon. Hybridization of the probe DNA and target DNA is conducted using standard methods known in the art. In one embodiment, the hybridizations are conducted in a 50 ml tube and allowed to rotate freely in a hybridizer. Membranes are prehybridized in a prehybridization buffer for 30 minutes at 71° C. The amount of probe used will depend on the efficiency of the labeling method used and can be determined by one of ordinary skill in the art. In one embodiment, 10 μl of each probe are boiled for 5 minutes and immediately placed on ice. The probe is then placed in 2 mls of preheated hybridization buffer and the probe mixture is added to the membrane. The amount of time for hybridization and the ideal temperature will depend on the probe and the label used and can be determined by one of ordinary skill in the art. In one embodiment the probe is hybridized to the membrane for 1 hour at 71° C. After hybridization the membrane is washed using known methods in the art.

Detection of probe hybridization to the membrane will depend on the probe used and can be determined by one of skill in the art. When a digoxigenin label is used an example of a suitable detection method includes the use of an anti-digoxigenin-AP conjugate and the color solution, NBT/BCIP. The detection development time will depend on the detection method used and can be determine by one of skill in the art. The hybridization of the probe DNA to one or more target DNA spots on the membrane indicates the source or sources of fecal contamination in the water sample.

### EXAMPLE 1

### Rapid Detection of Human Fecal Contamination in Estuarine Environments by PCR targeting of Bifidobacteria adolescentis

Sample Sites and Collection:

Sewage was collected from the influent at the Milledgeville Municipal Sewage Treatment Plant. Fecal samples of various animals were collected in and around Baldwin County, Ga. The water samples were collected by the University of Georgia Marine Extension staff and sent to the lab on ice. Eight estuaries in Georgia were sampled: Black Bank Creek, Altamaha River, West Point—Fedrica River, Dunbar Creek, a tributary to the Little Satilla River, two tributaries to Turtle Head River, and the Little Satilla River. Dunbar Creek was chosen because there was a recent spillage of 50,000 gallons of raw sewage in this creek reported to The Georgia Department of Natural Resources on Jul. 6, 2005.

Enterococci counts:

All water and sewage samples were filtered using EPA approved membrane filtration, Method 1600 (30). One hundred ml from each sample site was filtered through a 0.45 μm GN-6 filter (Pall Corporation, Ann Arbor, Mich.). Enterococcal counts were determined for each of the eight sample sites on triplicate mEI agar plates incubated for 24 hours at 41° C. Any colonies displaying a blue halo were counted to determine the CFU (colony forming units) of each water sample. The CFU was used to establish the level of enterococci at the sampling sites and to give an estimate of the levels of microbial contamination found in the rivers.

DNA Extraction:

Sewage from the water treatment plant was collected and diluted to 1/10, 1/100, and 1/1000 in 0.9% saline solution. Triplicate 50 ml sewage samples were filtered through a 0.45 μm filter (GN-6—Pall Corporation, Ann Arbor, Mich.) or a 0.22 μm Type GS nitrocellulose filter (Millipore, Billerica, Mass.) to determine which filter yielded higher concentrations of DNA. One hundred ml of each of the estuarine water samples were filtered through a 0.22 μm Type GS nitrocellulose filter. The filters were placed in a Petri dish and held in a freezer at −20° C. until the DNA was extracted. The eight water samples and sewage dilutions were processed with the Ultraclean™ Soil DNA Kit (MO BIO Carlsbad, Calif.) using a modification of the “Alternative Protocol.” The bead solution was initially separated from the beads and placed in the Petri dish containing the filter from the estuarine water or sewage samples. Solutions S1 and IRS were placed directly in the Petri dish. The dish was then vortexed vigorously for 15 minutes. The solution was removed from the dish and returned to the 2ml volume bead tubes and vortexed a maximum speed for 10 minutes. The solution in the bead tubes was then centrifuged at 10,000×g for 30 seconds. The supernatant was then transferred to a clean micocentrifuge tubes and 250 μl of Solution S32 (MO BIO's DNA precipitation solution) was added. The tubes were vortexed for 5 seconds and then incubated at 4° C. for 5 minutes. The tubes were then vortexed at 10,000×g for 1 minute. Avoiding the resulting pellets, the entire volume from each tube was transferred to clean microcentrifuge tubes. To each tube, 1.3 ml of Solution S3 (MO BIO's DNA cleaning solution) was added and vortexed for 5 minutes. Approximately 700 μl from each tube was loaded onto a spin filter and centrifuged for 1 minute at 10,000×g. The flow through was discarded and the remaining supernatant was added to the spin filters and centrifuged at 10,000×g. This process was repeated until all of the supernatant had been loaded onto a spin filter. To each spin filter, 300 μl of Solution S4 (MO BIO's second DNA cleaning solution) was added and centrifuged for 30 seconds at 10,000×g. The flow through was discarded and the spin filters were centrifuged for an additional 1 minute at 10,000×g. The spin filter were then placed in a new clean tube and and 50 μl of Solution S5 (MO BIO's DNA elution solution) was added. The spin filters were then spun for 30 seconds at 10,000×g. The tubes then contained the isolated DNA in S5 and the spin filters were discarded.

In addition, DNA from cow, horse, dog, goose, pig, and chicken feces was extracted using a Ultraclean™ Fecal DNA Kit (MO BIO Carlsbad, Calif.). For each animal, 0.2 g of feces were added to the 2 ml Fecal Dry Bead tubes. To each tube 550 μl of Fecal Bead solution was added and gently vortexed to mix. This was followed by the addition of 60 μl of S1 to each tube and then each tube was inverted to mix. Then 200 μl of Solution IRS was added to each tube. The Fecal Bead tubes were then vortexed at maximum speed for 10 minutes. The Fecal Bead tubes were then centrifuged at 10,000×g for 30 seconds. The resulting supernatant was then transferred to a clean microcentrifuge tube. To each tube 250 μl of Solution S2 was added. The tubes were then vortexed for 5 seconds and incubated at 4° C. for 5 minutes. The tubes were then centrifuged for 1 minute at 10,000×g. Avoiding the resulting pellet, 450 μl of supernatant were transferred to clean microcentrifuge tubes. To each tube 900 μl of Solution S3 was added and vortexed for 5 seconds. Approximately 700 μl of the solution was then loaded onto a spin filter and centrifuged for 1 minute at 10,000×g. The flow through was discarded and the process was repeated until all of the remaining solution had been passed through a spin filter. To each spin filter 300 μl of Solution S4 was added and centrifuged for 30 seconds at 10,000×g. The flow through was discarded and the spin filters were centrifuged again at 10,000×g for 1 minute. The spin filters were then placed in clean microcentrifuge tubes and 50 μl of Solution S5 was added to the center of the spin filter. The spin filters were then centrifuged for 30 seconds at 10,000×g. The spin filters were discarded and the tubes then contained the isolated DNA in S5 solution. The concentrations of the extracted DNA samples were determined using a Nanodrop ND-1000 Spectrophotometer (Wilmington, Del.).

Positive and Negative Controls:

B. adolescentis genomic DNA ATCC® number 15703D™ was used as a positive control for the PCR procedure. Sewage from the sewage treatment facility which contains B. adolescentis from human feces was used as an environmental positive control and to optimize the PCR reactions. DNA extracts from cow, horse, dog, chicken, pig, and goose feces were used as negative controls since these animals are likely candidates for fecal contamination of rivers. Prior to the PCR with Bifidobacterium primers, all water samples were subjected to PCR using eubacterial primers 8F (SEQ ID NO:5) and 785R (SEQ ID NO:1) (Amann et al. Microbiol. Rev. (1995) 59:143-169) to establish that the DNA extracts of the water samples were suitable for PCR amplification.

PCR Protocols:

The specificity of bifidobacteria primers used in this study were verified using the BLAST database search program (www.ncbi.nlm.nih.gov/blast) (Altschul et al. Nucleic Acids Res. (1997) 25:3389-3402). A nested PCR protocol was performed on the extracted DNA samples. The first step consisted of an amplification using the universal bacterial 16S rDNA primer, 785R (SEQ ID NO:1) (Amann et al. (1995)) and the Bifidobacterium genus specific primer, IM26F (SEQ ID NO:2) (Langendijk et al. Appl. Environ. Microbiol. (1995) 61:3069-3075). Each PCR reaction contained a 50 μl volume with 0.3 mM dNTP, 4 mM MgCl₂, 1.5 U Taq DNA polymerase, 1×PCR reaction buffer, and ˜30 ng of template DNA. The samples were run on a TC-312 Thermal Cycler (Techne Cambridge, UK) under the following conditions: initial denaturing at 94° C. for 5 min; 30 cycles of 94° C. for 30s, 48° C. for 30s, and 72° C. for 30s; and final elongation at 72° C. for 5 min. Product from this PCR reaction was then used as the template for a second PCR mixture.

For the second PCR protocol, the template was amplified using B. adolescentis species specific primers ADO1 (SEQ ID NO:3) and ADO2 (SEQ ID NO: 4) (Bonjoch (2004)). One μl of product from the first PCR was added to a 50 μl reaction mixture containing the same concentrations of MgCl₂, reaction buffer, dNTP, and Taq as above. PCR was performed under the following conditions: initial denaturing at 94° C. for 5 min; 45 cycles of 94° C. for 30s, 48° C. for 20s, 55° C. for 20s, and 72° C. for 1 min; and final elongation at 72° C. for 5 min. Products from both the first and second PCR were subjected to electrophoresis in a 1.5% agarose gel stained with ethidium bromide and analyzed in a UVP GelDoc-IT Imaging System (UVP, Upland, Calif.) to detect the presence of the appropriate bands.

Cell Counts and DNA Recovery:

Enterococci in the water samples were enumerated on mEI agar to establish the level of microbial fecal contamination at each sample site. Bacterial colonies exhibiting a blue halo were counted as positive for enterococci. A tributary to the Little Satilla River had the highest levels of enterococci contamination. Dunbar Creek, which was contaminated with sewage a week before sampling, had unexpectedly low levels of enterococci and in the Altamaha River, enterococci were undetected (Table 1).

Initially bacteria cells from the sewage were collected on a 0.45 μm filter instead of the 0.22 μm filter. However, when using the 0.45 μm filters the DNA recovery was 20% lower than the DNA recovered with the 0.22 μm filter (data not shown). The DNA was extracted from each water sample using a modified procedure from the MoBio Ultraclean™ Soil DNA Kit. This extraction method was easy to perform and reliably extracted between 1.5 and 2.0 kg of DNA from the water samples. PCR amplification of the 16S rDNA in all the samples indicated that the extracted DNA was suitable for PCR use. Table 1 shows the values obtained for the DNA extractions from 100 ml of sample, the enterococcal counts, 16S rDNA amplification, and whether human fecal contamination was detected in each sample.

PCR Detection of Bifidobacteria adolescentis:

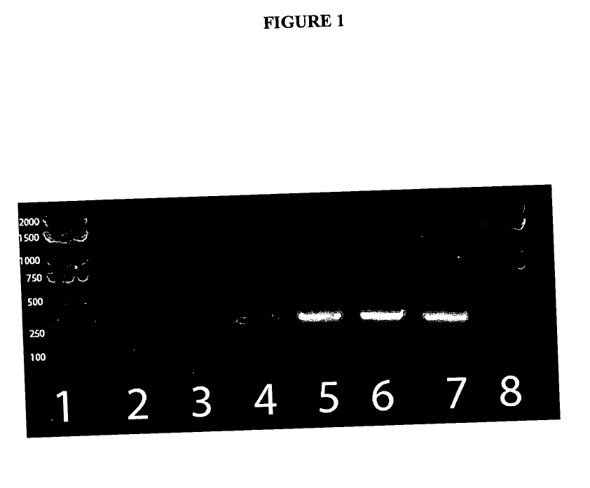

B. adolescentis DNA purchased from ATCC was used to establish the initial PCR conditions for the detection of B. adolescentis. The detection of B. adolescentis as a genetic marker of human fecal pollution in aquatic environments was optimized using sewage since human fecal bacteria were the intended targets. In DNA extracts of diluted sewage bacteria recovered on a 0.45 μm filter, the genetic marker was not detected in sewage dilutions higher than 1/10 (data not shown). Therefore we abandoned the use of the 0.45 μm filters in all subsequent experiments and filtered samples with the 0.22 μm filters. A critical step in the recovery of microbial DNA for subsequent PCR analysis was filtering the water using a 0.22 μm filter to collect the bacteria cells. The use of 0.22 μm filters in the procedure greatly increased the sensitivity of detection of the genetic marker. Optimum conditions were established when the B. adolescentis amplicon was detected after the second PCR protocol in a sewage sample diluted to at least 1/1,000. FIG. 1 shows the detection of a 279 bp genetic marker in diluted sewage samples which indicates the presence of B. adolescentis and human fecal microbial contamination. The maximum detection limit for the genetic marker was reached in DNA extracted from a 1/10,000 diluent of sewage. Fecal DNA extracts from dogs, cows, geese, horses, pigs, and chickens were used as negative controls. The 279 bp DNA fragment was not detected in any of the animal feces tested.

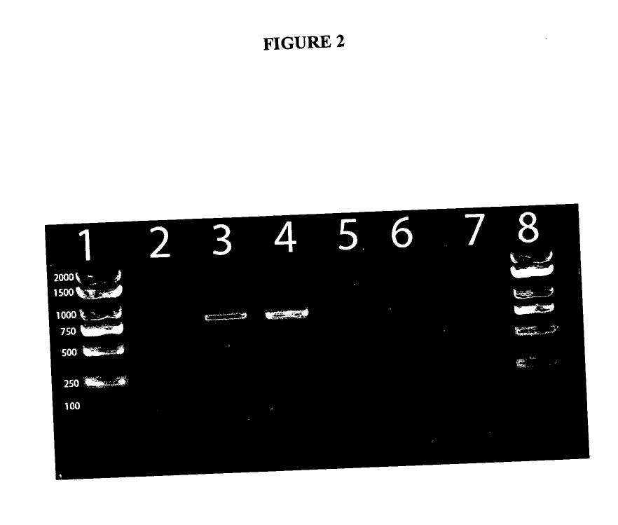

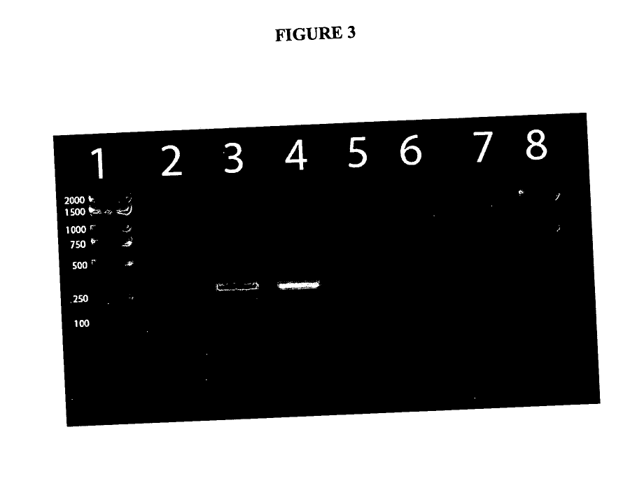

Each of the eight water samples was subjected to two PCR protocols. The presence of a 777 bp band on an agarose gel indicates the presence of bifidobacteria. A 777 bp band was detected in seven of the eight water samples as well as in the sewage and positive and negative controls. The negative control is expected to contain Bifidobacteria spp. and therefore be positive in this step of the method. In contrast, in the Altamaha River, an amplicon was not detected FIG. 2). The PCR product from the river samples and controls were then subjected to the second PCR protocol using the B. adolescentis species specific primers. FIG. 3 indicates that the positive control, sewage, and Dunbar Creek samples displayed the genetic marker of 279 bp. Of the other rivers tested, Black Bank Creek and a tributary to the Little Satilla River also produced an amplicon of this size (Table 1). In contrast, the negative control, Altamaha River, West Point—Fedrica River, Little Satilla River, and two different tributaries to the Turtle River did not produce the genetic marker for the presence of human fecal pollution (FIG. 3, Table 1).

This study shows that the molecular detection of Bifidobacteria adolescentis can be used as an effective genetic marker of human fecal contamination in Georgia estuaries. Enterococci enumerations on mEI media indicated that a tributary to the Little Satilla River with 516 CFU/100 ml was the most polluted of all the rivers tested. Extracted DNA from eight river water samples was subjected to a two-step nested PCR protocol using genus and species specific primers for Bifidobacteria spp. and B. adolescentis. B. adolescentis was detected in Dunbar River, Black Bank Creek, and in Little Satilla River tributary which demonstrates the presence of human fecal contamination in these three rivers. In the five other estuaries tested including West Point—Federica River and the Altamaha River that had less than 16 CFU/100 ml of enterococci, B. adolescentis was not detected.

TABLE 1
Enterococci counts, along with the concentration of DNA
extracted from each water sample, detection of bacterial
16S rDNA, and if human fecal contamination was detected

| | | Average | Bacterial | Human |
|---|---|---|---|---|
| | Enterococcal | DNA | 16S | Fecal |
| Sample Site: | Count | Extracted | rDNA | Contami- |
| Estuaries | (CFUs/100 ml) | (ng/μl) | Detected | nation |
| Altamaha River | Too few to | 26.14 | Yes | No |
| Sound | detect | | | |
| West Point - | 16 ± 3.1 | 31.15 | Yes | No |
| Fedrica River | | | | |
| Dunbar Creek | 22 ± 2.9 | 39.75 | Yes | Yes |
| Turtle River | 55 ± 6 | 71.5 | Yes | No |
| Tributary - | | | | |
| Head Drive | | | | |
| Turtle River | 58 ± 3 | 9.01 | Yes | No |
| Tributary - | | | | |
| Highway 82 | | | | |
| Little Satilla | 86 ± 7 | 9.07 | Yes | No |
| River | | | | |
| Black Bank | 160 ± 3.7 | N/A | Yes | Yes |
| Creek | | | | |
| Little Satilla | 516 ± 17 | 97.0 | Yes | Yes |
| Tributary - | | | | |
| Buck Swamp | | | | |
| Road | | | | |

### EXAMPLE 2

### Identification of Non-Point sources of Animal Fecal Contamination Using Dot Blot Hybridization with Bifidobacterium

DNA Extraction:

DNA from the animal feces was extracted using the MoBio Ultraclean™ Fecal DNA Kit. 0.25 g of each animal feces were used following the manufacturer's supplied protocol yielding 50 μl of DNA. Several different samples of feces from each type of animal were collected and mixed before extraction. All DNA extractions were quantified using the Nanodrop ND-1000 spectrophotometer.

Target Preparation:

The targets for hybridization were made using different PCR protocols. In short, preparing the target consisted of an amplification of the extracted DNA from the animal feces or water samples using the specific primers for each gene below. After PCR, all samples were purified using the Qiagen QIAquick® PCR Purification Kit (Valencia, Calif.) and diluted to the appropriate concentrations.

PCR of Eubacterial 16S rDNA:

To produce the two target regions of DNA consisting of the 16S rDNA of all eubacteria, two sets of bacterial primers were used. To produce the 777 bp amplicon, primers 8F (SEQ ID NO: 5) and 785R (SEQ ID NO: 1) were used, while primers 8F and 338R (SEQ ID NO: 6) (Brosius et al. J. Mol. Biol. (1981) 148:107-127) were used to produce the 330 bp amplicon. Each PCR reaction contained a 50 μl reaction volume with 0.2 mM dNTP, 3 mM MgCl₂, 1.5 U Taq DNA polymerase, 1×PCR reaction buffer, and ˜30 ng of template DNA. All samples were run on a Thermo Electron Corporation PxE0.2 Thermal Cycler (Waltham, Mass.) under the following conditions: initial denaturing at 94° C. for 5 min; 30 cycles of 94° C. for 30s, 52° C. for 30s, and 72° C. for 30s; and final elongation at 72° C. for 5 min.

PCR of bifidobacteria 16S rDNA:

To produce target DNA consisting of the 16S rDNA from bifidobacteria, the extracted DNA was amplified using the 16S rDNA primer, 785R (SEQ ID NO: 1) and the bifidobacteria genus specific primer, IM26F (SEQ ID NO: 2) (Langendijk et al. (1995)). Each PCR reaction contained a 50 μl reaction volume with 0.3 mM dNTP, 3 mM MgCl₂, 1.5 U Taq DNA polymerase, 1×PCR reaction buffer, and ˜30 ng of template DNA. All samples were run on a Thermo Electron Corporation PxE0.2 Thermal Cycler under the following conditions: initial denaturing at 94° C. for 5 min; 30 cycles of 94° C. for 30s, 48° C. for 30s, and 72° C. for 30s; and final elongation at 72° C. for 5 min.

Probe Preparation:

All probes were labeled nonradioactively using digoxigen (DIG) following the manufacturers recommended protocol (Boehringer Mannheim, Indianapolis, Ind.). When making the probes, 0.15 μl of 25 nmol Digoxigenin-11-dUTP was added to the PCR reaction mixture. This produced a probe that was then purified with the Qiagen QIAquick® PCR Purification Kit. The probe was diluted using autoclaved distilled water to a final concentration of 1 ng/μl.

Membrane Preparation:

All hybridizations were performed on the GeneScreen Plus® Hybridization Transfer Membrane (PerkinElmer, Boston, Mass.). To prepare the membrane, the target DNA from the animal feces was boiled and immediately placed in an ice bath. 2 μl dots of each of the extracted animal feces DNA were then placed on the membrane in concentrations of 5 ng/μl and 2.5 ng/μl. This resulted in dots containing 10 ng and 5 ng of animal fecal DNA respectively. The membrane was cross-linked in a Spectroline® UV Crosslinker at 1.2×10⁵ J/cm² (Spectroline, Westbury, N.Y.). The membrane was the then briefly washed in autoclaved distilled water before being stored at 4° C. until used.

DNA-DNA Hybridization:

For hybridization, the membrane was placed in a 50 ml tube to allow it to rotate freely in the UVP HB-1000 Hybridizer (Upland, Calif.). All membranes underwent prehybridization in DIG Easy Hyb (Roche) for 30 minutes at 71° C. 10 μl of each probe was boiled for 5 minutes and immediately placed on ice. The probe was then placed in 2 ml of the preheated DIG Easy Hyb and the probe mixture was added to the membrane. The probe was allowed to hybridize for 1 hour at 71° C. After hybridization, the membrane was washed twice for 5 min in 25 ml 2×SSC, 0.1% SDS at 20° C. Next, the membrane was washed twice for 20 min in preheated 0.5×SSC, 0.1% SDS at 75° C. The membrane was finally washed for 5 min in 25 ml washing buffer at 20° C.

Detection of DIG-labeled DNA:

Detection of the probe hybridized to the membrane was performed using the antibody solution, Anti-Digoxigenin-AP Conjugate, and the color-substrate solution, NBT/BCIP (Roche). The membrane was then added to 5 ml of detection buffer (0.1 M Tris-HCL, 0.1 M NaCl, pH 9.5 (20° C.)) and allowed to develop in the dark. Usual developing time was approximately 4 hours before the dots appeared. Hybridizations were done in triplicates with different collected fecal samples from each animal type.

DNA-DNA Hybridization with Eubacterial Probes:

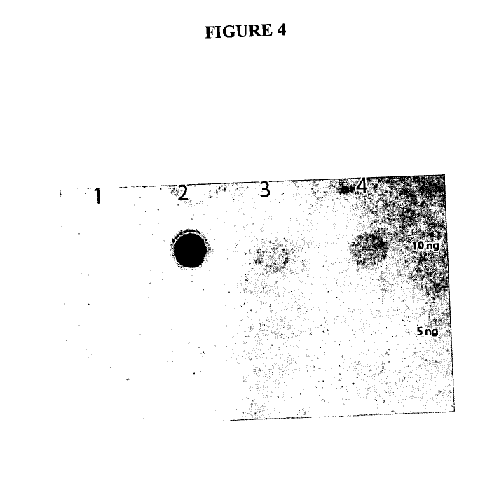

The use of a DIG labeled probe from the 16S rDNA of eubacteria found in the feces of dogs, pigs, geese, and cattle consistently produced cross reaction between the probe and all fecal targets on the membrane. This cross reaction was seen in both the 777 and 330 bp probes used. FIG. 4 shows the results and the nonspecific nature of a dog fecal probe made from this gene. The dog probe matched the dog fecal DNA on the membrane; however, the probe also hybridized to the feces of the other animals present on the membrane.

DNA-DNA Hybridization with Dog and Pig Fecal Bifidobacteria Probe:

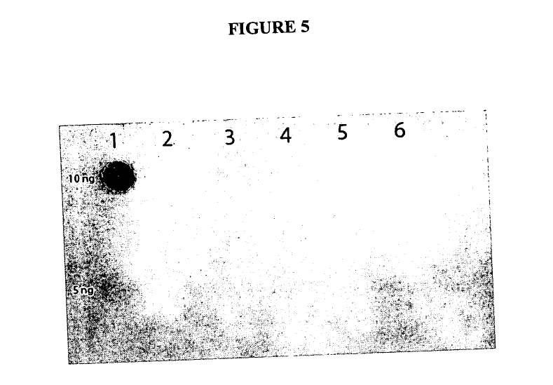

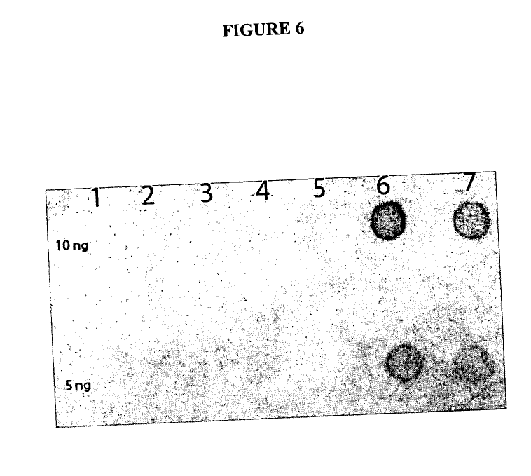

The 16S rDNA probe made from the 16S rDNA of bifidobacteria from dog feces produced no cross reaction with other animal DNA present on the membrane. The 777 bp size probe consistently provided accurate fecal identification on the membrane. This can be seen in FIG. 5. In addition, the pig fecal bifidobacteria probe also produced no cross reaction in the multiple trials performed. Probes made from the 16S rDNA gene of bifidobacteria present in pig fecal samples consistently hybridized to the pig fecal DNA present on the membrane. FIG. 6 shows DNA extracts from pig used as a probe for target identification.

Conclusion:

The results of this study show that DNA-hybridization using probes derived from Bifidobacterium DNA isolated from environmental water samples can be used to identify non-point sources of fecal contamination. PCR amplicons of bacterial genes from various animal feces, including dogs, cattle, geese, horses, chickens, and pigs, were tested to identify potential sources of fecal pollution. Based on the results of these test, it appears that the source of fecal pollution can be rapidly identified by DNA hybridization of fecal bacterial DNA gene targets against DNA-probes obtained from the environmental sample. Through methods such as these, the sources of microbial contamination in the environment can be determined, and appropriate steps taken to eliminate the problem.

I claim:

1. A method for detecting human fecal contamination in environmental water samples comprising;
a) filtration of water sample and capture of bacteria on filter membrane;
b) isolation of bacterial DNA directly from solid adsorbent;
c) amplification of Bifidobacterium genus specific portion of 16S rRNA gene from DNA isolated in (b);
d) amplification of Bifidobacterium adolescentis specific portion of 16S rRNA gene from DNA amplified in (c); and
e) detection of amplified DNA in (d), wherein the detection of the amplified DNA indicates the presence of human fecal contamination in the water sample and the lack of detection indicates the absence of human fecal contamination in the water sample.

2. A method for determining the source of fecal contamination in environmental water samples comprising;
a) isolation of bacteria from water sample;
b) extraction of DNA from bacteria isolated in (a);
c) amplification and labeling with a detectable marker of a Bifidobacterium genus specific portion of 16S rRNA gene from DNA isolated in (b);
d) hybridization of amplified and labeled DNA from (c) to a membrane containing fecal DNA isolated from potential animal sources of fecal contamination, wherein the DNA isolated from the fecal sample of each animal is spotted on a specific and separate spot on the membrane;
e) detection of hybridization of DNA from (c) to membrane; wherein detection of hybridization with fecal DNA of a given species indicates the source or sources of fecal contamination.


SEQUENCE LISTING (8 sequences)

SEQ ID NO: 1 - DNA, 19 bp, Eubacteria
ctaccagggtatctaatcc

SEQ ID NO: 2 - DNA, 21 bp, Bifidobacterium spp.
gattctggctcaggatgaacg

SEQ ID NO: 3 - DNA, 18 bp, Bifidobacterium adolecentis
ctccagttggatgcatgt

SEQ ID NO: 4 - DNA, 17 bp, Bifidobacterium adolecentis
cgaaggttgctcccagt

SEQ ID NO: 5 - DNA, 20 bp, Eubacteria
agagtttgatcctggctcag

SEQ ID NO: 6 - DNA, 18 bp, Eubacteria
gctgcctcccgtaggagt

SEQ ID NO: 7 - DNA, 19 bp, Eubacteria
gagttcatcaactaccgca

SEQ ID NO: 8 - DNA, 16 bp, Eubacteria
cttccaccacggtgtc